(19) United States
(12) Patent Application Publication
(10) Pub. No.: US 2022/0212022 A1
(43) Pub. Date: Jul. 7, 2022

(54) METHOD AND APPARATUS FOR SELECTIVE MODULATION OF NEURONAL FUNCTION AND OF AND TISSUE PERMEABILITY WITH MRI CORRELATION

(71) Applicant: Weinberg Medical Physics, Inc., North Bethesda, MD (US)

(72) Inventors: Irving N. WEINBERG, Chevy Chase, MD (US); Oleg UDALOV, North Potomac, MD (US); Sahar Jafari CHAMKAVI, North Bethesda, MD (US); Ittai BAUM, Silver Spring, MD (US)

(21) Appl. No.: 17/568,122

(22) Filed: Jan. 4, 2022

Related U.S. Application Data
(60) Provisional application No. 63/133,629, filed on Jan. 4, 2021.
(60) Provisional application No. 63/225,834, filed on Jul. 26, 2021.

(51) Int. Cl. A61N 2/00; A61N 2/02; A61N 2/06

(52) U.S. Cl. CPC A61N 2/006; A61N 2/06; A61N 2/02

(57) ABSTRACT

Apparatuses and methods safely apply electric fields deep in a body by selectively actuating multiple magnetic modules about the body sequentially in time to modulate tissue. The electric field induced in regions of the body from such actuations may have a different frequency, depending on the depth of the region.

### CROSS REFERENCE AND PRIORITY CLAIM

This patent application claims priority to U.S. Provisional Application No. 63/133,629 entitled “METHOD AND APPARATUS FOR SELECTIVE NERVE MODULATION WITH MRI CORRELATION,” filed Jan. 4, 2021, and U.S. Provisional Application No. 63/225,834 entitled “APPARATUS AND METHOD FOR SELECTIVELY MODULATING THE BLOOD BRAIN BARRIER WITH TOMOGRAPHIC ELECTROPERMANENT MAGNET ARRAYS,” filed Jul. 26, 2021, both of which being incorporated by reference herein in their entireties.

### FIELD

Disclosed embodiments pertain to methods and apparatuses for modulating nerve- and neuron-containing structures, and barriers in animals, including humans. In particular, disclosed embodiments relate to apparatuses and methods for selective modulation with tomographic electropermanent magnet arrays that may be used in medical and veterinary applications.

### BACKGROUND

The brain and several other organs (e.g., spinal cord, ovaries, testes, eyes, inner ears) normally maintain barriers against penetration by large molecules or organisms. This barrier property can be used to advantage in delivering drugs or genes to specific locations in these organs, for example, by selectively opening the barrier in those specific locations without opening the barrier throughout the organ. For the purposes of this disclosure, all such barriers in all such organs will be referred to when the term blood-brain barrier, or “BBB” is used, even when the organ is not the brain. As taught by L. Huang in the 2020 article entitled: “Challenges in adeno-associated virus-based treatment of central nervous system diseases through systemic injection”, researchers are developing viral vectors for gene therapy capable of generating new neuronal tissues, and the performance of these vectors might be improved if they were delivered in high concentrations to targeted regions in the brain.

The term permeability is used to indicate transport characteristics into tissue, for example across a blood-brain barrier or within a cancer that has reduced flow as a result of disordered vascular supply.

Focused UltraSound (“FUS”) has been promoted as a method for increasing the permeability across (“opening”) the BBB, but there are some challenges, including damage to neurons even at low pulse strengths, as taught by Z. I. Kovacs et al, in the 2016 article entitled “Disrupting the blood-brain barrier by focused ultrasound induces sterile inflammation.”

It is known that transient electric fields may open the BBB, as taught by S. Sharabi et al, in the 2019 article in Drug Delivery, entitled “Transient blood-brain barrier disruption is induced by low pulsed electrical fields in vitro: an analysis of permeability and trans-endothelial electric resistivity”. The application of focal pulsed electric fields in the brain may be performed with electrodes, as disclosed by Sharabi. The effect of pulsed electric fields on the BBB may depend on both amplitude and frequency.

Pulsed electric fields in the brain may be induced by transient magnetic fields that are applied non-invasively, as in Transcranial Magnetic Stimulation (TMS). Conventional TMS instruments are not able to generate strong electric fields deep in the brain, as taught by Z. De Deng, S. H. Lisanby, and A. V. Peterchev, in the 2014 Clinical Neurophysiology article entitled “Coil design considerations for deep transcranial magnetic stimulation.” This inability is unfortunate, because some locations of particular interest to physicians and researchers are deep in the brain, for example, hippocampal circuits responsible for memory. TMS applies changing magnetic fields to the surface of the head. The changing magnetic fields induce electric fields in the surface of the brain. These magnetic fields fall off rapidly with depth in the body, so that conventional TMS systems are unable to directly stimulate such structures.

It is known that bi-phasic transient electric fields induced by magnetic fields can open the BBB, as taught by S. Heydarheydari et al in a 2021 article in Electromagnetic Biopsy and Medicine. Heydarheydari employed a conventional TMS coil, which creates a biphasic electrical field in the brain. The term bi-phasic electrical field is used to mean an initial pulse of electrical field of one polarity followed by a pulse of opposite polarity with similar duration, as defined by J. P. Reilly, V. T. Freeman, and W. D. Larkin in the 1985 publication in the IEEE Transactions on Biomedical Engineering Vol. BME-32, No, 12, entitled “Sensory Effects of Transient Electrical Stimulation—Evaluation with a Neuroelectric Model”.

### SUMMARY

Disclosed apparatuses and methods safely apply electric fields deep in a body by selectively actuating multiple magnetic modules about the body sequentially in time. The electric field induced in regions of the body from such actuations may have a different frequency and waveform, depending on the depth of the region. Since the likelihood of nerve modulation is dependent on the rate of change (i.e., frequency) and waveform of the electric field, nerves in different regions may be differentially modulated. This principle can be used to apply transcranial magnetic stimulation or cardiac stimulation deep in the body.

In some embodiments, disclosed apparatuses and methods deliver payloads such as genes or other large molecules to organs with barriers, for example, the brain. Non-invasive tomographic application of magnetic fields generated by multiple coils, electromagnets or electropermanent magnets are used to transiently generate electric fields in these organs. The electric fields transiently open the barriers to payloads. The application of the magnetic fields may be accompanied by imaging using the same or similar multiple coils, electromagnets, or electropermanent magnets, the imaging being used to facilitate accurate localization and electric field dosimetry for payload delivery.

In at least some embodiments an apparatus and method is provided for delivering pulsed electric fields in specified locations of the brain and other organs, for the purpose of opening barriers in these locations so that desired therapeutic or other agents may be selectively delivered to those locations or so that noxious substances may be removed (for example, by the circulation) from those locations.

### BRIEF DESCRIPTION OF THE FIGURES

FIG. 1 shows an apparatus having an assembly of modules partially surrounding a body part;

FIG. 2 illustrates two configurations of the assembly of FIG. 1 partially surrounding body part and corresponding graphs show one component of vector potential spatial distribution in a certain slice of the body in FIG. 2B, as well as the electric field as a function of time near the surface of the body part and deep in the body part as the magnetic configurations are changed from as a function of time in FIG. 2C.

FIG. 3 illustrates how selective actuation of modules in the assembly of FIG. 1 may rotate the vector potential in space as a function of time;

FIG. 4 illustrates how the regions of electrical field may be changed in time through changes in module activation so as to yield a smaller region of neuronal excitation according to the disclosed embodiments;

FIG. 5 is a flow chart illustrating an embodiment of the method by applying configurations as in FIGS. 2-4, sometimes at the same time.

FIG. 6 is a flow chart illustrating an embodiment of the method in which a magnetic resonance image examine whether a region of modulation is appropriate for neuronal modulation before the modulation has been applied;

FIG. 7 illustrates an example of a human or other animal at various times in the vicinity of an assembly of FIG. 1 having least a coil, electromagnet, or electropermanent magnet;

FIG. 8 is a block diagram of a control system and current source according to the disclosed embodiments;

FIG. 9 is a flow chart of a method of modulating barriers according to the disclosed embodiments; and

FIG. 10 illustrates electric fields generated by an electropermanent magnet with unsaturated and saturated cores.

### DETAILED DESCRIPTION

Reference is made to the U.S. patent application Ser. No. 17/358,696 incorporated by reference herein, entitled “METHOD AND APPARATUS FOR MODULATION OF TRACTS IN NERVOUS TISSUE.”

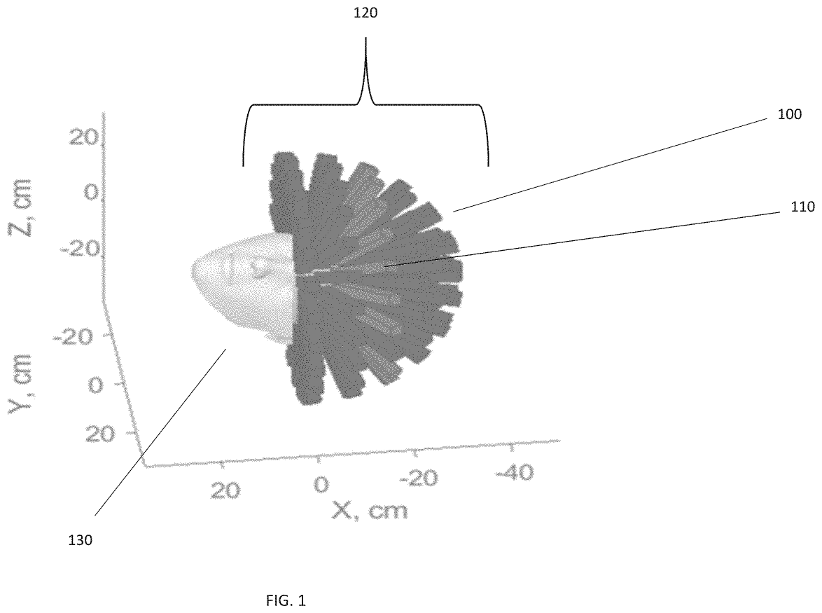

Disclosed embodiments include an apparatus as illustrated in FIG. 1, having an assembly 120 of at least two magnetic modules 100 and 110. Each module 100, 110 contains magnetizable material or another source of magnetic fields. It is understood that in FIG. 1, magnetic modules 100 and 110 are shown as examples of modules, and they may be of different shapes, sizes, or configurations. In some embodiments, each magnetic module may constitute an electropermanent magnet, containing a core of AlNiCo or other magnetizable rods or other shapes surrounded in part by one or more coils of electrically conductive material. The magnetic module may be actuated by causing electrical currents to flow in the one or more coils. The magnetic module may have one or more magnetic sensors for adjustment or monitoring of the magnetic field created by the magnetic module or other magnetic modules in the assembly. For the purposes of this description, the terms “modules” and “magnetic modules” are used interchangeably. In some embodiments, the magnetic module may contain coils without a core to create a magnetic field. One or more magnetic modules in the assembly may be actuated (“fired’) at a different time than other magnetic modules in the assembly. For example, module ring 110 is shown in a different shade of gray, indicating that its actuation state is different than the rest of the modules. The assembly may surround or be near a body part 130. By “near”, it is meant less than 100 cm in distance. The body part may be a brain of an animal such as a human or rat. The body part may be a heart or other structure containing nerves or neurons or other tissue that may be physiologically affected by magnetic or electrical fields. Such physiological effect may include modulation, stimulation, inhibition, transmission, or blocked transmission of nerve impulses. The magnetic module may include a coil or other sensor for transmission or reception of radiofrequency energy from a body part.

For the purposes of this description, the terms “neuron”, “neuronal”, “nerve” and “neuronal tissue” are used interchangeably to mean tissue containing neurons or nerves or tissues stimulated or otherwise affected by neurons or nerves (for example, muscles or muscle groups in the heart, gut, inner ear, optic nerve, nerve root or other locations). The term “stimulation” is intended to mean increasing the firing frequency, but is also used to mean any modulation, for example, interference with transmission or inhibition of impulses conveyed by nerves or neurons. The term “actuation” is used interchangeable with “activation” and means the changing of the magnetic state of one or more magnetic modules.

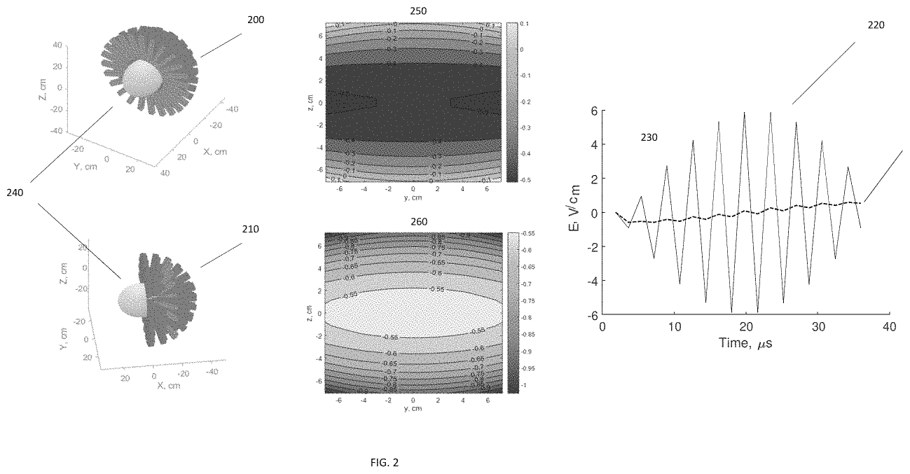

FIG. 2 shows two actuation configurations of assembly 120 partially surrounding body part 240. As one or more sets of magnetic modules (examples of sets being 200 and 210) fire differently with respect to the rest of the assembly, electrical fields are induced in the body part 240. The different sets of magnetic modules fired in 200 and 210 create electrical fields in the shallow portion of the body part that vary in time as the modules are fired, as shown in trace 220 in FIG. 2C. Deep in the body part, the electrical fields change more slowly in time, as shown in trace 230. Such a difference in the timescale of electric field application in the superficial and deeps area is achieved through a chosen set of actuated magnets and chosen extent of actuation for each magnet. An example of vector potential spatial distribution (one component only) is shown in FIG. 2B in graphs 250, 260 for actuation configurations 200, 210, respectively. Two actuation configurations may be chosen in such a way that in both of them the vector potential in the deep region is similar, while the vector potential at the superficial region is very different. Therefore, in an embodiment, electric field quickly oscillates at the surface and slowly varies in the middle by slowly increasing and decreasing the actuation strength.

An important aspect of the disclosed embodiments is that they exploit a physiological phenomenon, in which the effect of changing electrical fields applied to neuronal or nervous tissue is highly dependent on the duration and waveform of the change. This phenomenon was described by Weinberg et al in the publication entitled “Increasing the oscillation frequency of strong magnetic fields above 101 kHz significantly raises peripheral nerve excitation thresholds.,” published in Medical physics, vol. 39, no. 5, pp. 2578-83, May 2012, doi: 10.1118/1.3702775. As discussed in that publication, nerves have a much higher threshold for stimulation when magnetic fields with rise or fall times of less than 10 microseconds (or with frequencies greater than 101 kHz) are applied to the nerves, as compared to magnetic fields that rise or fall more slowly. Thus the invention permits the selective modulation of deep structures. It should be understood that modulation or stimulation may not be “all or nothing” states, and so the selective modulation may be the modulation to a lesser or greater extent. For purposes of this description, the term frequency is used interchangeably with rise and fall time, whereby a high frequency means that the rise and/or fall time is short, and a low frequency means that the rise and/or fall time is long. For example, a 100 kHz frequency pulse sequence implies that the rise time is about 10 microseconds and the fall time is about 10 microseconds.

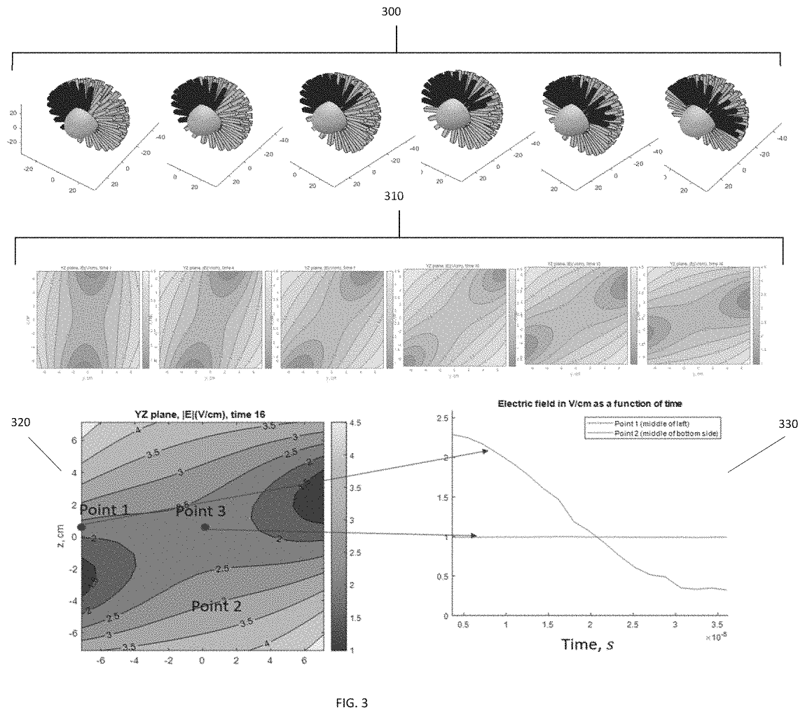

FIG. 3 illustrates another embodiment of the invention, in which the location and extent of the electric field modulation region is established. Activated magnets for several different points in time 300 are shown with darker color 302. The activated sector 302 of magnetic modules rotates at each next operation. The absolute value of the vector potential spatial distribution is illustrated 310. By successively selecting different sets of modules for magnetization 300, the vector potential, and therefore magnetic and electric fields 310, can rotate in the body part, and the electric field at a superficial portion of the body part, such aspoint 1 in 320, changes in time at a different rate as seen in graph 330, than in a deep location in the body part illustrated at point 3 in graph 330.

Embodiments shown in FIGS. 2 and 3 can be combined or interleaved to achieve sophisticated control of neuronal excitation.

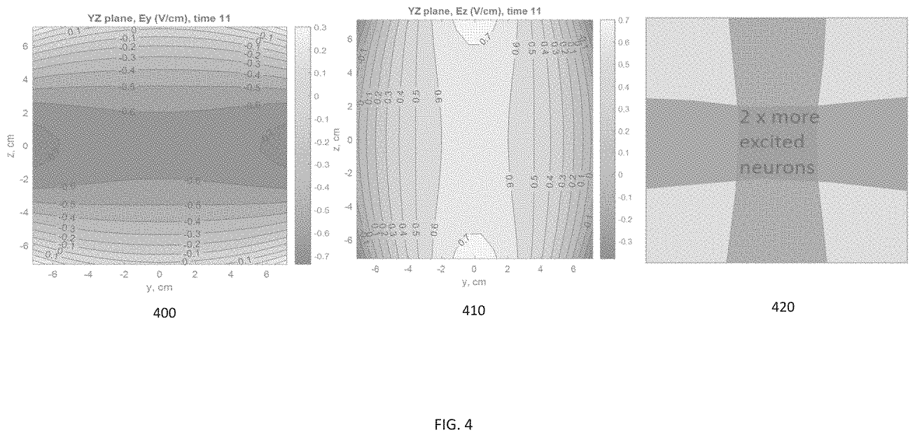

FIG. 4 illustrates how the regions of electrical field may be changed in time through changes in module activation so as to yield a smaller region of neuronal excitation. Illustration 400 shows the electrical field caused by one configuration of activated modules. Soon thereafter (for example, within 50 microseconds) the configuration is changed to 410. The region of expected neuronal activation is shown in 420.

FIG. 4 shows that by successively selecting different sets of modules for magnetization to yield different distributions 400 and 410 of high electric field, a smaller region 420 of nervous tissue can be excited. Electric field distribution 400, for example, may be activated and soon thereafter (for example, within 50 microseconds) the configuration is changes to cause electric field by second configuration 410. It is known that a rotating electric field is more efficient at stimulating neurons, as taught by Y. Roth et al in the article entitled “Rotational field TMS: Comparison with conventional TMS . . . ” published in Brain Stimulation 13 pages 900-907 (2020). Modules can be selected that achieve a rotational electric field as shown in FIGS. 3 and 4.

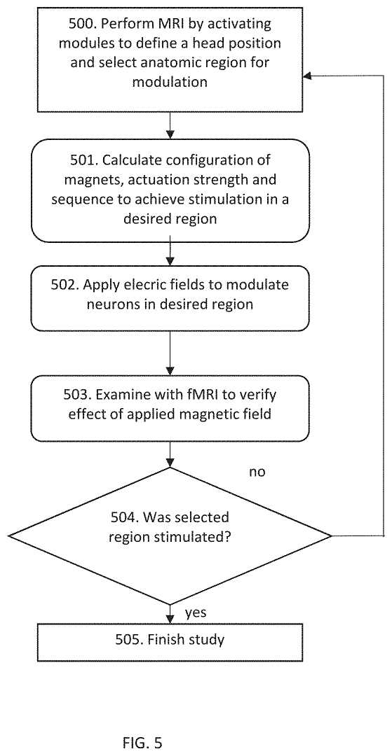

FIG. 5 demonstrates a method of modulating a selected region as applied to a patient or subject (human or otherwise). Magnetic resonance images may be collected 500 via MRI, for example, by using the modules in the disclosed assemblies in conjunction with radiofrequency antennas and/or gradient coils (not shown). The position of one or more selected regions in the body of the patient or subject may be used to determine which magnetic modules to actuate, and with what degree of magnetization.

A calculation is performed to determine which magnets and in what sequence should be activated to stimulate a desired region 501. Modules are actuated to stimulate or modulate neurons in the selected region of interest 502. An MRI may be used with appropriate pulse sequences to collect functional images 503, for example, examining blood flow in the region (which generally is affected by neuronal stimulation). The images collected in 503 can be examined by a person or computer to verify that the TMS had the desired effect 504. If not, then the method may perform MRI to define head position and select anotomic region for modulation500 may be repeated. It should be understood that many repetitions of various operations may be done until the study is ended 505.

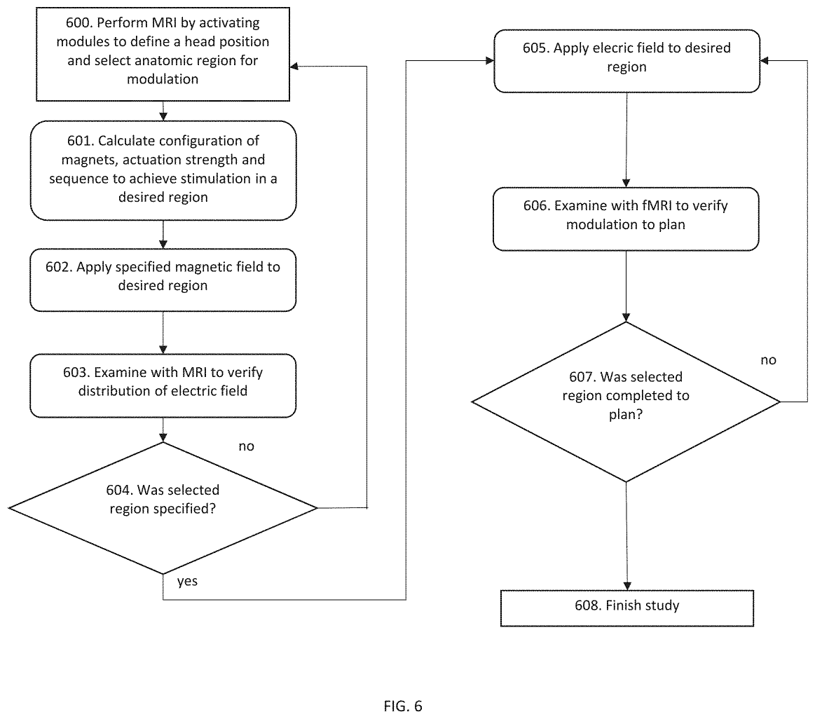

FIG. 6 illustrates a method in which a magnetic resonance image is collected to verify correct region specification prior to application of modulation. An MRI is obtained by actuating magnetic modules to assist in localizing a region for modulation 600. A calculation is made (that may include data collected in operation 600) of the appropriate sequence of actuation of magnetic modules to modulate a region in a body part 601. A magnetic field is created 602 in the body part according to the calculation 601. An MRI pulse sequence is executed by actuating magnetic modules 603. The results of the MRI performed are analyzed to verify that the correct region has been selected according to plan 604. If so, an electric field may be applied to the desired region 605 . If not, some or all of operations 600-604 may be repeated. Mang modules are actuated to apply an electric field to the selected region. An MRI pulse sequence may be executed to examine whether neuronal tissue in the selected region was modulated 606. This pulse sequence uses blood flow, blood volume or other functional MRI techniques for the purpose of determining whether modulation has been according to plan. The MRI is analyzed to seen if the plan has been executed correctly, and whether the planned number of repetitions has been executed 607. If so, operation 608 concludes the study. If not, some or all of operations 600-607 may be repeated.

It should be understood that some of the operations in FIG. 5 or 6 may be omitted or be placed in different orders, and that sets of magnetic modules actuated in some operations may differ from the sets of magnetic modules actuated in other operations. It should be understood that the operations may be interrupted or otherwise changed according to the status of the animal subject (for example, in case of seizures). It should be understood that the subject may undergo multiple sessions of treatment with the operations of FIG. 5 or 6, for example, repeating the sessions weekly as needed to reduce symptoms of a disease.

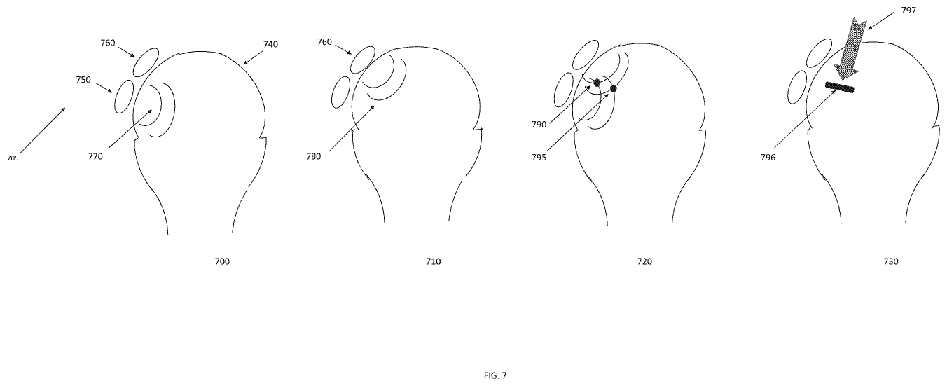

FIG. 7 shows another example of one embodiment of the disclosed apparatus 705. The head of a human or other animal 740 is shown at various times 700, 710, 720, 730. It should be understood that the head might be replaced by some other organ or organs with barriers, and the term “head” is used in this specification to represent one of such multiple possible organs.

At least one coil or electromagnet or electropermanent magnet 750 is in the vicinity (i.e., within one meter) of the head 740. At least one additional such coil or electromagnet or electropermanent magnet 760 is similarly located in the vicinity (i.e., within one meter) of the head 740. At time corresponding to 700, coil 750 is activated by a current from a system to generate magnetic field 770 in head 740.

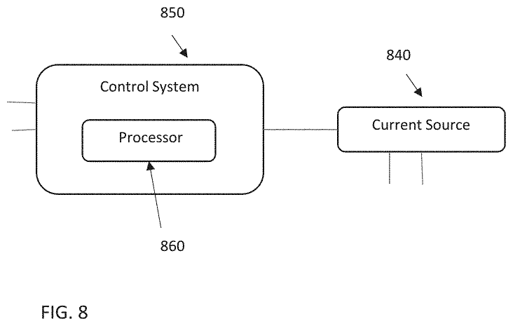

The source of the current 840 and the control system 850, including a processor 860, are illustrated in FIG. 8, which can connect via wires or wirelessly to the apparatus 705.

At a subsequent time (that may be shorter than a neuronal response time for example, less than 0.1 seconds, less than 100 milliseconds, less than 500 microseconds as described in U.S. Pat. No. 9,411,030 incorporated by reference), the apparatus may generate a current through coil 760 to generate magnetic field 780 in head, as shown in 710. Time 720 represents the magnetic field as perceived by the brain, in which case the physiological effects of the electric fields induced by the magnetic fields of times 700 and 710 are integrated or otherwise combined at locations 790 and 795. As shown in time 730, the summed effects may modulate the barrier in location or tract 796 in the brain (or in another organ with a barrier).

The terms “modulate” and “modulation” may be used to indicate relative increased permeability of a barrier to payloads (“opening”), or decreased permeability, as desired by the practitioner or researcher. The term “intended location” or “intended region” may refer to and include a location or region in the organ with a barrier. The term “response” may refer to and indicate the onset and/or duration of modulation.

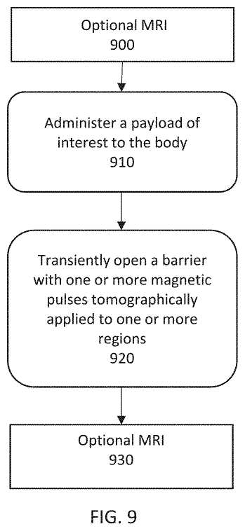

The above operations in one example of a method are illustrated in the flow chart of FIG. 9. Although the term “subsequent operation” is used in the next section of this specification for illustration of the method of the invention, it should be understood that some operations may be in different orders and may be repeated. The sequence may start at 900 with an MRI of the brain or other organ with a barrier (e.g., spinal cord).

The sequence may continue with administration 910 of an agent whose selective administration to an organ with a barrier is to be accomplished with this method. For example, the agent may be a virus intended for gene therapy at specific locations in the brain. The administration may be systemic (for example, with an intravenous or oral injection) or may be through another route (for example, into the cerebrospinal circulation).

At least two current pulses are sent through one or more coil(s) or electromagnet(s) or electropermanent magnet(s) 920, with phases selected so that the electric fields produced by the pulses result in modulation of the barrier at a pre-determined location in the organ.

A magnetic resonance (MR) image may be obtained 930 to determine the magnetic fields generated in any or all of the above operations. This determination may include examination of effect of the magnetic fields on the spin states at various locations and times, for example, as taught by D. E. Bohning et al., “Mapping transcranial magnetic stimulation (TMS) fields in vivo with MRI,” NeuroReport, vol. 8, no. 11, pp. 2535-2538, 1997, doi: 10.1097/00001756-199707280-00023.

As discussed above in the description of the Figures, the apparatus 705 comprise at least two coils or electromagnets or electropermanent magnets 750 and 760 within one meter of neuronal or nervous tissue such as brain, or of tissue in another organ with a barrier 740. It should be understood that the terms “neuronal” or “nervous” tissue refers to tissues containing nerves or neurons. In FIG. 7, the organ is represented as a brain-containing head 740.

It should be understood that the term “electropermanent magnet” includes an apparatus including at least one coil or current-carrying material (for example, a wire) and magnetizable material, wherein the magnetization of the magnetizable material changes in magnitude or direction as a result of the current, and at least some of this change in magnetization persists after a current is run through the at least one coil or current-carrying material. For the purposes of this disclosure, the term “coil” includes coils, portions of current-carrying material, electromagnets and electropermanent magnets. For the purposes of this disclosure, the phrase “at least two coils configured to be arranged in the vicinity of an organ with a barrier and to generate electric fields for modulation of the barrier” is meant to include generation of magnetic fields by the coil, the magnetizable material, or the combination of both the coil and the magnetizable material. For the purposes of this disclosure, the term “core” is used to describe the magnetizable material within the electropermanent magnet.

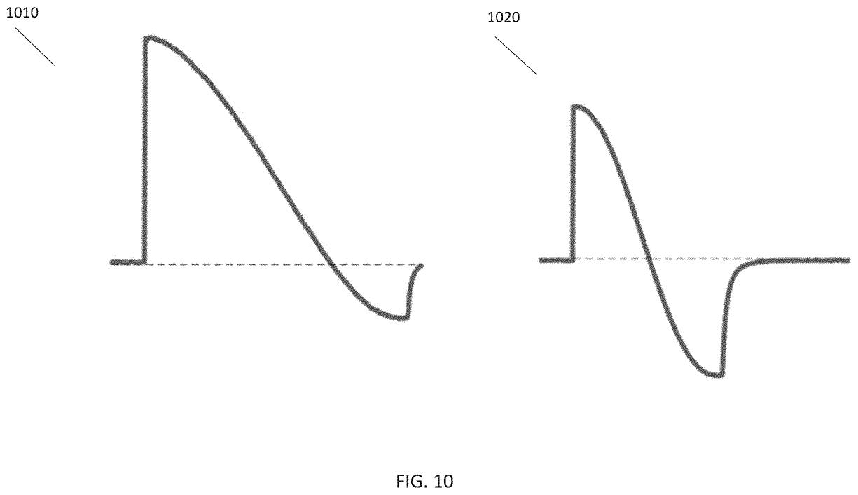

FIG. 10 illustrates how the initial magnetic state of the magnetizable material in an electropermanent magnet may influence the electrical field produced by the electropermanent magnet in response to a supplied current (for example, a discharging capacitor). Electric field probe trace 1010 is an illustration of the predominantly mono-phasic electric field generated by an electropermanent magnet, in which the initial state of the core was relatively unsaturated (for example, at zero magnetization). Electric field probe trace 1020 is an illustration of the predominantly bi-phasic electric field generated by an electropermanent magnet, in which the core was initially closer to magnetic saturation than in the case 1010.

In accordance with disclosed embodiments, the coils 150 and 160 and other possible coils may be activated with electrical currents to create electric fields in tissues at one or more intended locations in tissues within the subject's organ (or organs) the organs and/or tissues containing a barrier in order to modulate the barrier.

For example, the intended locations may be the hippocampi in a human brain of a patient with Alzheimer disease, and the barrier may be opened to admit genetic vectors that program tissue to regenerate cells. As another example, the intended locations may be substantia nigra in a patient with Parkinson disease, and the barrier may be opened to admit genetic vectors that program tissue to regenerate cells or to generate dopamine.

As another example, the intended locations may be plaques in a patient with Alzheimer disease, and the barrier may be opened to the vascular circulation to assist in removing such plaques.

The electrical currents to the various coils may be generated at sequential times so that the electric fields sum, subtract, modulate, or otherwise combine over a region in the organ that is larger or different than the region of any one coil. This combinatory effect is akin to tomography, in the sense that electric fields at the intended location formed by the combination is different than the electric field that would be caused by any single coil. Unlike x-ray tomography, in which the combinations of x-ray beams are only additive, the use of different coils with different phases allows the combination of electric fields at the intended regions to be additive, subtractive, or to create electric fields at different effective magnitudes and effective frequencies at the intended and other regions as compared to the electric fields that would be generated by an single coil.

An aspect of the invention is that the frequencies and/or amplitudes of the electric fields at specific intended locations may be different than at other locations, which property may be useful so as not to painfully stimulate or otherwise effect regions (for example, the scalp) other than the intended targets, the intended targets containing barriers.

The direction of the electric effect at the intended location may be controlled by changing the time, phase, and magnitude of currents through the coils. It should be understood that the term “magnetic effect” may include the induction of electric fields that may stimulate, inhibit, or otherwise modulate physiological activities of tissues within the organ. The physiological effect of the magnetic effect on tissue in the organ is denoted “physiological effect”, which may include modulation of the barrier within the tissues and may also include neuronal tissue stimulation or inhibition.

For the purposes of this disclosure, the terms “integrate” and “integration” are meant to refer to and describe operations wherein the response time of the barrier within the tissue (e.g., the integration time) to successive magnetic or electrical effects is less than the time between the start and/or end of each magnetic or electrical effect, or is less than the time between the start and end of all magnetic effects. The integration time may include the time for a single neuron or nerve or tissue to respond to modulation, the time for a group or neurons or nerves or tissue to respond to modulation, or the time for a circuit containing a group of neurons or nerves or tissues to respond to modulation.

For the purposes of this disclosure, the term “integration time” is meant to be comparable in length with neuronal or tissue response times. Neuronal response times may be several milliseconds, or tens or milliseconds, hundreds of milliseconds, or longer, depending on the type of neuron or nerve or tissue and the number of neurons or nerves or tissues involved in the response.

The terms “integrate” and “integration” also are meant to include partial overlap or minimal separation (for example, less than 1 second) of the magnetic effects in time, to establish a perceived summation, relative motion and/or direction of successive magnetic effects at various locations. Such integration may be used to generate arbitrary shapes of barrier opening in the organ. As an example, if a magnetic effect is applied to a subject's brain by a coil (for example, coil 750 at the location of coil 750) and then another magnetic effect is applied by another coil (for example, coil 760 at the location of coil 760), and these magnetic effects overlapped within an integration time, then a circuit in the brain may be modulated (via physiological effect) as if both coils 750 and 760 were activated at the same time at their respective locations. It should be understood that this process may be applied to create physiological effects with many sizes and shapes. For example, FIG. 7 shows how summations of the magnetic effects caused by regions of overlapping magnetic or electric fields at locations 790 and 795 could produce a region of opened barrier 196, through which material 797 could flow.

If the magnetic effects of the two or more coils were not applied at the same time, then the physiological effect may be directional within a neuronal pathway or tract or tissue. Adjustments in the phase of the magnetic fields generated by the coils may be used to further specify the size, magnitude, location, or direction of such modulation. It should be understood that the use of MR or magnetic particle imaging may assist in making such adjustments, for example, through a feedback routine that examines the magnetic fields through MR or magnetic particle imaging and then changes the magnetic fields.

The use of a magnetizable magnetic core within one or more electropermanent magnet(s) provides an advantage for the invention in that said magnetic core may be magnetically polarized (or depolarized) prior to the application of electrical current, so that a predominantly mono-phasic electric pulse is induced, as illustrated in FIG. 10. Thus the magnetization state of the core of an electropermanent magnet may be used to control the electric field waveform produced at least in part by the electropermanent magnet. As taught by Reilly, a predominantly mono-phasic pulse is more effective than a bi-phasic pulse in modulating neuron activity. It is understood that there is a spectrum between bi-phasic and mono-phasic pulses, which is why the term “predominantly” is used.

For the purposes of this disclosure, a predominantly mono-phasic electric field may be defined as having an electrical field pulse magnitude that is at least 25% higher than the magnitude of a next electric field pulse of opposite polarity. In an embodiment, a predominantly mono-phasic electric field may be defined as having an electrical field pulse magnitude that is at least 50% higher than the magnitude of a next electric field pulse of opposite polarity.

Prior work has used electromagnets without cores to generate predominantly mono-phasic pulses, by having electrical currents travel in opposite directions with different time-scales, as taught in the 2011 J Neural Eng. article (volume 8, number 3) by A. V. Peterchev, D. L. Murphy, and S. H. Lisanby entitled “Repetitive Transcranial Magnetic Stimulator with Controllable Pulse Parameters”.

For the purposes of this disclosure, the degree of phase of the electric field pulse (e.g., predominantly mono-phasic versus predominantly bi-phasic) for each location is included in the term “electric field waveform” or “electrical field waveform”, or simply “waveform”.

Operations in the method are shown in FIG. 9. One or more imaging studies 900 and 930 may be obtained prior to, during, and/or after activation of the coils, in order to add information concerning the spatial extent or other description of the applied magnetic fields (for example, as performed by Bohning who examined the effect on spins in the organ caused by applied TMS pulses), and of the response of nervous tissue (for example, with the BOLD effect) or other tissues (for example, detecting passage of magnetic contrast material). The imaging study may be obtained with magnetic resonance imaging, electron resonance imaging, magnetic particle imaging, ultrasound imaging, or other means that may or may not employ the electropermanent magnets used to perform the modulation. It should be understood that the electropermanent magnets may be used to collect images, so that TMS and imaging may be performed without moving the subject from one platform to another. An advantage of the invention is that without such motion, position accuracy is better and the timing between barrier modulation and MRI is reduced so that the measurement of the response of tissue is more accurate. The use of electropermanent magnets to collect images was previously described in issued U.S. Pat. No. 10,908,240 and in related patents including US Pat. Pub. 20170227617, entitled “METHOD AND APPARATUS FOR MANIPU LA TING ELECTROPERMANENT MAGNETS FOR MAGNETIC RESONANCE IMAGING AND IMAGE GUIDED THERAPY, incorporated herein by reference.

For the purpose of this disclosure, the terms “electric field” and “magnetic field” are used interchangeably, since a pulsed magnetic field generated by the apparatus creates both a magnetic field and an electric field in the organ. Measuring the size of a magnetic field created by the invention (for example, through MRI), when the timing of such creation is known, provides a description of the electric field induced in that region by the magnetic field.

It should be understood that the use of electropermanent magnets or electromagnets or coils in a single apparatus to generate the electric fields for modulation of barriers and to demonstrate anatomy and physiological responses of the tissue and to describe the extent of the magnetic fields generated to modulate the nervous tissue is novel. Since the one or more coils or electropermanent magnets may be turned on and off or otherwise modulated so that there is no static magnetic field being applied at the time that the modulation pulse is applied by one or more coils or electropermanent magnets, there will be no force on the one or more coils. Without the attendant motion that may accompany forces, such an apparatus may be safer to use than the system described by Bohning et al. Thus, the lack of a permanent static field provides additional safety and flexibility to the user of the apparatus.

It should be understood that the combinatory use of multiple electropermanent magnets with appropriate phasing as described herein may be more effective in modulating tissues that are deep in the organ as compared to the one to four electromagnet coils presently used in TMS.

It should be understood that magnetic pulse sequences for neuromodulation (for example, stimulation) or for assessment of neurological response (for example, BOLD), may follow, precede, or be interleaved with the magnetic pulse sequences for barrier modulation at intended locations.

It should be understood that the timing and activation magnitudes of multiple coils may represent an advantage in flexibility for opening the BBB over TMS devices that have specific configurations optimized to generate electric fields in specific regions. As an example, a user may choose to use the apparatus to modulate one region in a subject, and then modulate a different region in the same or another subject, without having to physically move the coil locations, but only having to change the electrical parameters (for example, the phase and/or magnitude) of the currents supplied to the at least two coils in the invention. The invention therefore corresponds to an “all-purpose” system that might be used by a medical practitioner or researcher to treat or otherwise affect many conditions.

The use of multiple functionalities of the apparatus may be useful in achieving a desired therapeutic effect. For example, opening a barrier to a therapeutic agent with the apparatus and following the opening with neurostimulation may increase the effectiveness of that agent. In this case, the invention results in underproduction (or reduced concentration) of the undesired cancer cells.

It should be understood that the apparatus and method of the invention may be used in cancer to deliver a payload to the location of cancer or to other locations where metastasis from the cancer or growth of the cancer may be reduced. In this case, the invention may result in increased production and/or concentration of the desired cells in the tissue, for example, macrophages programmed to kill cancer cells. An example of magnetic field application using a TMS coil was disclosed in the 2019 J Biomed Phys Eng publication by B. Yousefian, S. M. P. Firoozabadi, and M. Mokhtari-Dizaji entitled “Magnetoporation: New Method for Permeabilization of Cancerous Cells to Hydrophilic Drugs”. Differences between the present disclosed embodiments and this prior work include the ability of the present embodiments to (1) deliver predominantly mono-phasic electric fields, (2) create waveforms of said electric fields to create neuromodulation or permeability in desired locations in the body, (3) obtain images to prescribe the location of the electric field, and (4) obtain images to verify delivery of the magnetic field.

It should be understood that the apparatus and method of the invention may be used in conditions where a region in an organ with a barrier is deficient in producing a desired substance (for example, the substantia nigra in a patient with Parkinson disease) to deliver a payload (for example, a virus capable of gene therapy) to a location in an organ with a barrier where production and/or concentration of a desired substance by cells from that location may be increased.

It should be understood that the payload may be a molecule (e.g., chemotherapy drug), or a cell (e.g., a pre-programmed killer T-cell) or a physical change (for example, application heat) whose efficacy may be varied by modulation of the barrier.

It should be understood that the apparatus and method of the invention may be used in conditions where a region in an organ with a barrier is overproducing an undesired substance (for example, amyloid in a patient with Alzheimer disease) to deliver a payload (for example, a virus capable of gene therapy) to a location in an organ with a barrier where production (or concentration) of the undesired substance by cells from that location may be decreased.

Moreover, those skilled in the art will recognize, upon consideration of the above teachings, that the above exemplary embodiments and the control system may be based upon use of one or more programmed processors programmed with a suitable computer program. However, the disclosed embodiments could be implemented using hardware component equivalents such as special purpose hardware and/or dedicated processors. Similarly, general purpose computers, microprocessor based computers, micro-controllers, optical computers, analog computers, dedicated processors, application specific circuits and/or dedicated hard wired logic may be used to construct alternative equivalent embodiments.

Moreover, it should be understood that control and cooperation of the above-described components may be provided using software instructions that may be stored in a tangible, non-transitory storage device such as a non-transitory computer readable storage device storing instructions which, when executed on one or more programmed processors, carry out he above-described method operations and resulting functionality. In this case, the term “non-transitory” is intended to preclude transmitted signals and propagating waves, but not storage devices that are erasable or dependent upon power sources to retain information.

Those skilled in the art will appreciate, upon consideration of the above teachings, that the program operations and processes and associated data used to implement certain of the embodiments described above can be implemented using disc storage as well as other forms of storage devices including, but not limited to non-transitory storage media (where non-transitory is intended only to preclude propagating signals and not signals which are transitory in that they are erased by removal of power or explicit acts of erasure) such as for example, Read Only Memory (ROM) devices, Random Access Memory (RAM) devices, network memory devices, optical storage elements, magnetic storage elements, magneto-optical storage elements, flash memory, core memory and/or other equivalent volatile and non-volatile storage technologies without departing from certain embodiments. Such alternative storage devices should be considered equivalents.

It should be understood that the terms actuation or firing may represent partial or complete magnetization of one or more parts of one or more modules, with polarizations in one or more directions (e.g. South-North or North-South).

It should be understood that selection of the initial magnetization state of the core of an electropermanent magnet may be used to control the electric field waveform subsequently produced at least in part by the electropermanent magnet, for example to create a predominantly mono-phasic electric field.

It should be understood that arcs or other patterns in the neuronal tissue may be created with the apparatus and method, which may be helpful in diagnosis or treatment or research into connected parts of the neuronal tissue.

It should be understood that the region of interest may be within the assembly (for example, the region may be in the brain) or may be near the assembly (for example, the region may be in the heart).

It should be understood that the apparatus and method may be useful in treating patients with illnesses relating to abnormal function of structures deep in the brain, for example, addiction, anxiety, depression, or of other neurological diseases (for example, epilepsy).

It should be understood that electric fields generated in deep or other selected structures with the invention may be used to cure or otherwise treat or diagnose diseases such as cancer.

It should be understood that the ability to generate electrical fields at various locations with different frequencies may be useful aside from stimulation, for example, to selectively actuate administered materials (for example, magnetoelectric particles).

While various exemplary embodiments have been described above, it should be understood that they have been presented by way of example only, and not limitation. Thus, the breadth and scope of the present invention should not be limited by any of the above-described exemplary embodiments but should instead be defined only in accordance with the following claims and their equivalents.

For example, in accordance with some embodiments, an apparatus may comprise at least two coils configured to be arranged in the vicinity of tissue in an organ with a barrier and to generate magnetic or electric fields for modulation of tissue within the intended region(s) in the organ, and a control system configured to control timing of magnetic or electric fields applied for modulation of the tissue wherein the timing is within the integration time of the nervous tissue.

Optionally, the modulation created by the electrical fields generated by the at least two coils in combination may be different in size, shape, direction, or magnitude than the modulation that each coil creates separately. Optionally, the at least two coils may be within electropermanent magnets. Optionally, the at least two coils may be configured to be arranged within 1 meter of the nervous tissue. Optionally, the control system may be configured to activate a second coil of the at least two coils subsequent to activation of a first coil of the at least two coils. Optionally, the first coil and the second coil are configured to generate overlapping electrical fields in intended locations within the organ tissue. Optionally, the integration time may be less than 1 second and, for example, may be several milliseconds, or tens or milliseconds, or hundreds of milliseconds, or longer.

Optionally, the apparatus may be, further configured, by selecting electrical parameters of the at least two coils, to modulate one or more intended regions in one subject, and to modulate one or more different regions in the same or different subject or a different subject by adjusting the electrical parameters of the at least two coils. Optionally, the apparatus may be configured to both collect data used to form an image of the intended tissue in a subject and to modulate the intended tissue, without moving the subject.

In accordance with at least some embodiments a method of modulating nervous tissue may include positioning at least two coils in the vicinity of nervous tissue, generating magnetic fields by activating the at least two coils, and applying the magnetic fields to modulate the tissue in the organ, wherein timing of magnetic fields applied for modulation of the tissue is within the integration time of the tissue. Optionally, the magnetic fields from the at least two coils may be used to perform an imaging study before, after or during the modulation. Optionally, the at least two coils may be positioned within 1 meter of the nervous tissue.

Optionally, a second coil of the at least two coils may be activated subsequent to activation of a first coil of the at least two coils. The first coil and the second coil may generate overlapping electric fields in the tissue in the intended location(s). Again, here, optionally, the integration time is less than 1 second and, more particularly, the integration time may be several milliseconds, or tens or milliseconds, or hundreds of milliseconds, or longer.

The method may further comprise modulating one or more regions in one subject and then modulating one or more different regions in the same or different subject or a different subject. The magnetic fields applied to both collect data used to form an image of the tissue in a subject and to modulate the tissue, may be applied without moving the subject.

In some embodiments, an apparatus comprises an assembly of at least two magnetic modules, whereby at least one of the modules may be fired independently of another module, whereby the assembly induces electric fields at different frequencies at different locations in a body part, the body part being near or partially or totally enclosed by the assembly.

Optionally, the neural tissue in the body part may have a threshold for stimulation that is dependent on frequency so that deep sections of the body part may be stimulated or otherwise modulated to a different degree than other sections of the body part.

The magnetic module may include a magnetizable material and a coil of electrically conductive material. The body part may be the brain, nerve root, heart, inner ear, or gut of an animal.

In some embodiments, a method of selectively modulating neuronal tissue in a body part may comprise selectively actuating magnetic modules in an assembly near to or enclosing the body part, wherein the selective actuation creates electric fields at different frequencies at different locations in the body part.

The neural tissue in the body part may have a threshold for stimulation that is dependent on frequency so that deep sections of the body part may be stimulated or otherwise modulated to a different degree than other sections of the body part by the electric fields.

Optionally, the selective actuation results in modulation of an arc or other pathway in the body part.

Magnetic resonance imaging may be collected before or after the selective modulation to depict the localization of the electric fields. Transcranial magnetic stimulation may be applied to deep structures in the brain that are responsible at least in part for psychiatric, neurological, or addiction disorders of diseases. Electric fields may be applied to structures at selective locations in the body for diagnosis or treatment of cancers or other diseases. Electric fields may be applied to administered materials at selective locations in the body whereby the administered materials are actuated for diagnosis or treatment of diseases.

1. An apparatus comprising:
an assembly of at least two magnetic modules, wherein at least one of the modules may be fired independently of another module, and
a control system configured to control timing of electric fields generated by the assembly to induce electric fields at different frequencies or waveforms at different locations in a body part and to thereby modulate tissue differently at different locations in the body part.

2. The apparatus as in claim 1, where the each of the at least two magnetic modules includes a magnetizable material and a coil of electrically conductive material.

3. The apparatus of claim 2, wherein the magnetizable material comprises electropermanent magnets.

4. The apparatus of claim 1, where in the at least two electromagnetic modules are configured to generate overlapping fields the tissue.

5. The apparatus of claim 1, wherein the control system is configured to control the timing of the electric fields to modulate a barrier in the tissue of the body part, wherein the modulation caused by the size, frequency, magnitude, or direction of the electric fields is different than the modulation caused by each of the at least two modules separately.

6. The apparatus of claim 1, wherein neural tissue in the body part has a threshold for stimulation that is dependent on electrical field frequency or waveform so that deep sections of the body part are stimulated or otherwise modulated to a different degree than other sections of the body part.

7. The apparatus of claim 1, wherein the control system is configured to activate a second module of the at least two modules subsequent to activation of a first module of the at least two modules.

8. The apparatus of claim 7, wherein the first module and the second module are configured to generate overlapping electric fields in the tissue.

9. The apparatus of claim 1, configured to collect data used to form an image of the tissue in the subject and to modulate the tissue, without moving the subject.

10. The apparatus of claim 1, configured to deliver a payload to tissue that causes production of desired substances in the tissue or will cause regeneration of cells in the tissue.

11. A method of selectively modulating tissue in a body part comprising:
selectively actuating at least two magnetic modules in an assembly near to or enclosing the body part,
wherein the selective actuation creates electric fields at different frequencies or waveforms at different locations in said body part to selectively modulate tissues at different locations in said body part.

12. The method of claim 11, further comprising administering a payload to a subject with the body part, and applying the magnetic fields to modulate a barrier of tissue of the body part, wherein timing of the magnetic fields applied for modulation of the barrier of the tissue causes a different modulation than a modulation generated by applying a magnetic field of any individual module, and wherein the modulation of the barrier affects delivery of the payload to the tissue or of transport of a substance from the tissue.

13. The method of claim 11, further comprising activating the second magnetic module subsequent to activating the first magnetic module of the at least two modules.

14. The method of claim 11,wherein the at least two modules generate overlapping electric fields.

15. The method of claim 11, further comprising modulating one or more regions in one subject and then modulating one or more different regions in the same or a different subject.

16. The method of claim 11 further comprising performing an MRI with the at least two magnetic modules to select an anatomic region for modulation.

17. The method of claim 11, further comprising calculating a configuration of the magnetic modules, actuation strength and actuation sequence to achieve stimulation in a desired anatomic region.

18. The method of claim 11, further comprising applying electric fields to modulate neurons in the desired anatomic region.

19. The method of claim 11, wherein magnetic resonance imaging is collected before or after the selective modulation to depict the localization of the electric fields.

20. The method of claim 11, wherein selection of an initial magnetization state of the core of an electropermanent magnet is used to control the electric field waveform subsequently produced at least in part by the electropermanent magnet.

21. The method of claim 20, where the waveform of the electric field is predominantly mono-phasic.
